(19) United States
(12) Patent Application Publication
(10) Pub. No.: US 2017/0049153 A1
(43) Pub. Date: Feb. 23, 2017

(54) ELECTRONIC CIGARETTE/VAPORIZER AND ATOMIZER THEREOF

(71) Applicants: David Guo, Rancho Cucamonga, CA (US); Hongxi Guo, Rancho Cucamonga, CA (US)

(72) Inventors: David Guo, Rancho Cucamonga, CA (US); Hongxi Guo, Rancho Cucamonga, CA (US)

(21) Appl. No.: 15/242,213

(22) Filed: Aug. 19, 2016

Related U.S. Application Data
(60) Provisional application No. 62/206,990, filed on Aug. 19, 2015.

(51) Int. Cl. A24F 47/00; F16J 15/02; A61M 11/04; H05B 3/18

(52) U.S. Cl. CPC A24F 47/008; H05B 3/18; F16J 15/022; A61M 11/041; H05B 2203/021; A61M 2205/0211

(57) ABSTRACT

An atomizer for use with vaporizers and electronic cigarettes, the atomizer comprising an inhaler having a first end and a second end through both of which air flow passage partially extends; a chamber coupled to said second end of said inhaler; a heating element positioned within the chamber to be in contact with a liquid; a liquid absorber coupled to the heating element; and a receiver to receive the heating element, wherein the receiver has at least one conductive contact, and the receiver comprises a power connection connectable to a power supply.

### PRIORITY

This application claims priority to U.S. Provisional Application No. 62/206,990, entitled “ELECTRONIC CIGARETTE/VAPORIZER/PERSONAL VAPORIZER AND CERAMIZER THEREOF,” filed on Aug. 19, 2015, the entire disclosure of which is hereby incorporated by reference.

### FIELD OF THE DISCLOSURE

The field of the disclosure relates generally to electronic cigarettes, which are also referred to as vaporizers, or personal vaporizers, and various components therein.

### BACKGROUND OF THE DISCLOSURE

Conventionally, an electronic cigarette/vaporizer includes a cartridge and vaporizes e-liquid received in the cartridge by an atomizer. The atomizer usually includes a metal coil wrapped by cotton, and the e-liquid seeps onto the cotton wrapped coil. In use, the metal coil of the atomizer is heated to a high temperature which vaporizes the e-liquid.

However, the above design usually results in heavy metal poisoning to the user, because the vaporized e-liquid contains metal particles due to the high surface temperature of the heated metal coil. Thus, there is still a continuing need for improving the conventional electronic cigarette/vaporizer so as to eliminate the metal particles from the vaporized e-liquid.

United States Patent application publication number 20160157522 by Zhu discloses a vaporizer and electronic cigarettes having the vaporizer. Zhu was concerned with controlling the vapor flow, quantity and speed of e-liquid vaporization.

International Publication Number WO2016/005601 by Batista discloses an aerosol generating system comprising a removable heater. Batista was more specifically concerned with electric heaters becoming contaminated with material from aerosol-forming substrate during use.

All referenced patents, applications and literatures are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply. The embodiment may seek to satisfy one or more of the above-mentioned desires. Although the present embodiment may obviate one or more of the above-mentioned desires, it should be understood that some aspects of the embodiment might not necessarily obviate them.

Thus, there is still a need for improving the conventional electronic cigarette/vaporizer so as to eliminate the metal particles from the vaporized e-liquid.

### BRIEF SUMMARY OF THE DISCLOSURE

The present disclosure addresses and overcomes the above-described drawbacks of conventional electronic cigarette/vaporizer by providing an atomizer so as to eliminate the metal particles generated in the operation of the electronic cigarette/vaporizer. The disclosure is configured for all types of electronic cigarettes/personal vaporizers, so long as the electronic cigarettes/personal vaporizers requires a heating element to heat liquid contained therein. It is not necessary that the liquid be nicotine-based, the liquid may comprise one or more of a medicament, a tobacco derived material, and a flavorant. It is also not necessary that the invention requires a tank for holding the liquid. For example, it can be implemented in a dripper-type vaporizer where no tank is provided (user would drip liquid into the vaporizer every so often).

Among the many different possibilities contemplated, the atomizer of the embodiment may have a ceramic heating element electrically connecting to a power source; a liquid absorber wrapping around the ceramic heating element; a holder within a chamber coupling the liquid absorber and the heating element together; a receiver to receive the heating element and the liquid absorber, and wherein the chamber has openings communicating the inner and outer of the housing for the liquid absorber to absorb e-liquid.

In one embodiment, the heating element is made of tungsten embedded in aluminum nitride. In another embodiment, the clamp is electrically separated from the heating element. In yet another specific embodiment, the heating element is in a plate-shape having two opposite large surfaces. Various different shapes are contemplated, for example, flat configurations with circular shapes and rectangular shape, and cylindrical and polygonal configurations. The contemplated embodiments are designed to allow air to flow through various sides of the heating element, including flowing through the atomizer, and flowing through an opening in the chamber.

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWINGS

It should be noted that the drawing figures may be in simplified form and might not be to precise scale. In reference to the disclosure herein, for purposes of convenience and clarity only, directional terms such as top, bottom, left, right, up, down, over, above, below, beneath, rear, front, distal, and proximal are used with respect to the accompanying drawings. Such directional terms should not be construed to limit the scope of the embodiment in any manner.

FIG. 1 is a perspective view of an embodiment of an atomizer.

FIG. 2 is a side view of the embodiment of the atomizer of FIG. 1.

FIG. 3 is a top view of the embodiment of the atomizer of FIG. 1.

FIG. 4 is a bottom view of the embodiment of the atomizer of FIG. 1.

FIG. 5 is a cross sectional-side view of the embodiment of the atomizer of FIG. 1.

FIG. 6 is a perspective-exploded view of the embodiment of the atomizer of FIG. 1.

FIG. 7 is a cutaway-exploded view of the embodiment of the atomizer of FIG. 1.

FIG. 8 shows a perspective view of an embodiment of a housing of an atomizer removed from the chamber.

FIG. 9 shows an exploded-perspective view of an embodiment of the components within a housing of the atomizer of FIG. 8.

FIG. 10 is a top view of the embodiment of the atomizer of FIG. 8.

FIG. 11 is a bottom view of the embodiment of the atomizer of FIG. 8.

FIG. 12 is a cutaway-perspective view of the embodiment of the atomizer of FIG. 8.

FIG. 13 is a perspective view of the embodiment of a heating element of the atomizer.

FIG. 14 is a perspective view of another embodiment of an atomizer.

FIG. 15 is a cross sectional-side view of the embodiment of the atomizer in FIG. 14.

FIG. 16 is a perspective-exploded view of the embodiment of the atomizer in FIG. 14.

FIG. 17 is a side-exploded view of the embodiment of the atomizer in FIG. 14.

FIG. 18 is a perspective view of a base of the embodiment of the atomizer shown in FIG. 14.

FIG. 19 is a top view of a base of the embodiment of the atomizer shown in FIG. 14.

FIG. 20 is a cutaway-perspective view of a base of the embodiment of the atomizer shown in FIG. 14.

FIG. 21 is a perspective-exploded view showing a separated inhaler of the embodiment of the atomizer shown in FIG. 14.

FIG. 22 is a cutaway-perspective view of an inner part of the inhaler of the embodiment of the atomizer shown in FIG. 14.

FIG. 23 illustrates various views of a cap.

FIG. 24 is a transparent view of a cap.

FIG. 25 is a cross-section view of a cap.

FIG. 26 illustrates a locking mechanism in the cap.

FIG. 27 is a cross sectional view of another embodiment of the atomizer with a dripper.

FIG. 28 illustrates various views of a circular heating element capable of being used in a dripper-style atomizer or in a tank-style atomizer.

FIG. 29 is a perspective view of a circular heating element.

FIG. 30 is a perspective view of a dripper component.

FIG. 31 is an exploded cross sectional view of dripper atomizer components.

FIG. 32 is an exploded perspective view of dripper atomizer components.

FIG. 33 is a cross sectional view of another embodiment of the atomizer.

FIG. 34 is an exploded perspective view of the atomizer shown in FIG. 33.

FIG. 35 is an exploded cross sectional view of the atomizer shown in FIG. 33 where the heating element is detachably secured to the receiver.

FIG. 36 is an exploded cross sectional view of the atomizer shown in FIG. 33 where the heating element is detached from the receiver.

FIG. 37 is a perspective view of an embodiment of a heating element of the atomizer.

FIG. 38 is a perspective view of another embodiment of a heating element of the atomizer.

FIG. 39 is a perspective view of another embodiment of a heating element of the atomizer with multiple layers of trace partially overlapping.

FIG. 40 is various views of an easy fill embodiment.

FIG. 41 is various views of a seal within easy fill embodiment.

### DETAILED DESCRIPTION OF THE EMBODIMENT

The invention and its various embodiments can now be better understood by turning to the following detailed description of the preferred embodiments, which are presented as illustrated examples of the invention. It is expressly understood that the invention may be broader than the illustrated embodiments described below.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use must be understood as being generic to all possible meanings supported by the specification and by the word itself.

The definitions of the words or elements therefore include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements or that a single element may be substituted for two or more elements. Although elements may be described above as acting in certain combinations, it is to be expressly understood that one or more elements from a certain combination can in some cases be excised from the combination and that the combination may be directed to a subcombination or variation of a subcombination.

Thus, specific embodiments and applications of electronic cigarette/vaporizer with ceramizer are disclosed below. It should be apparent, however, to those skilled in the art that many more modifications besides those described herein are possible without departing from the inventive concepts herein. Moreover, in interpreting the specification, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Insubstantial changes from the subject matter as viewed by a person with ordinary skill in the art, now known or later devised, are expressly contemplated as an equivalent. Therefore, obvious substitutions now or later known to one with ordinary skill in the art are defined to be within the scope of the defined elements. The embodiment and its various embodiments can now be better understood by turning to the following detailed description of the embodiments, which are presented as illustrated examples of the embodiment defined in the claims. It is expressly understood that the embodiment as defined by the claims may be broader than the illustrated embodiments described below.

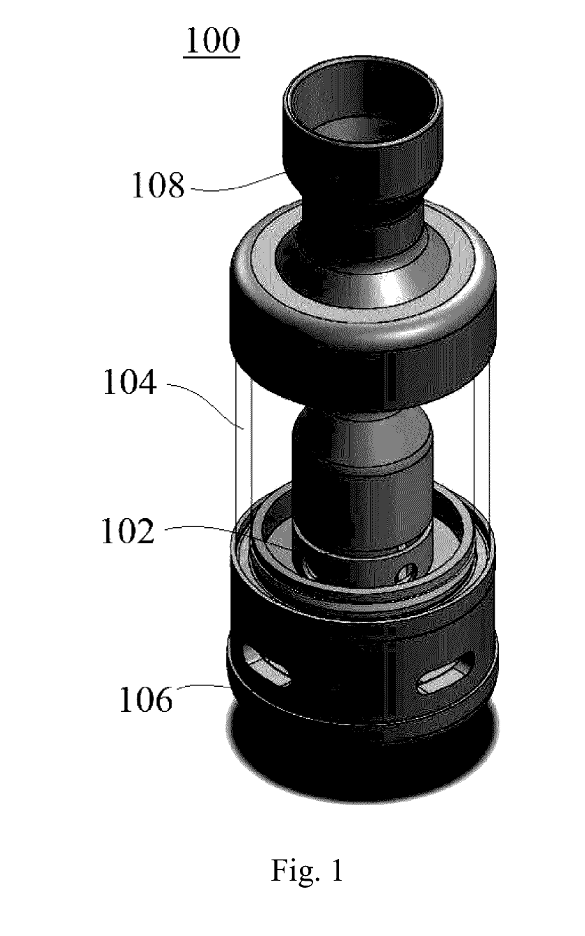

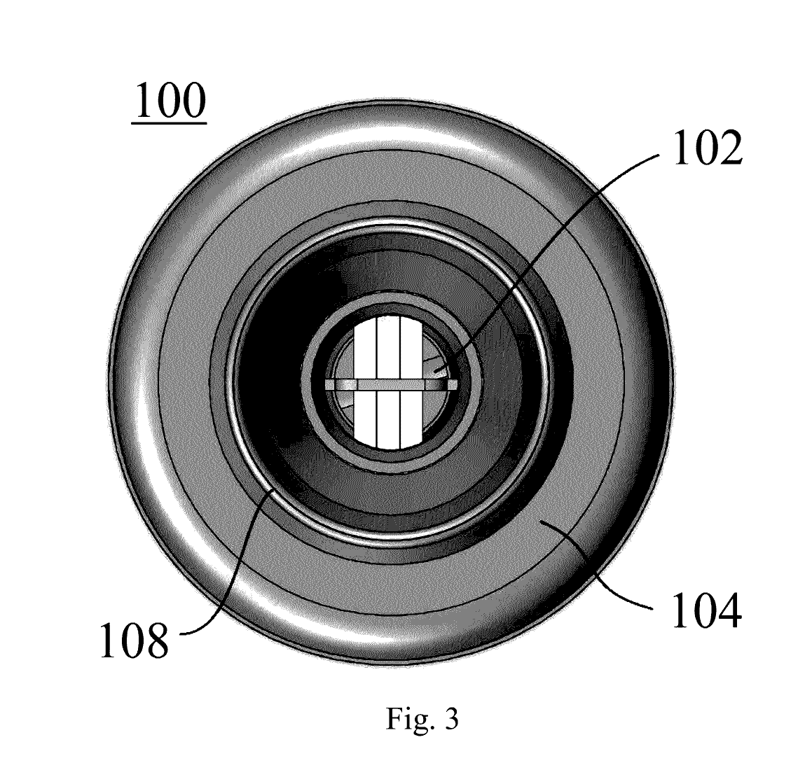

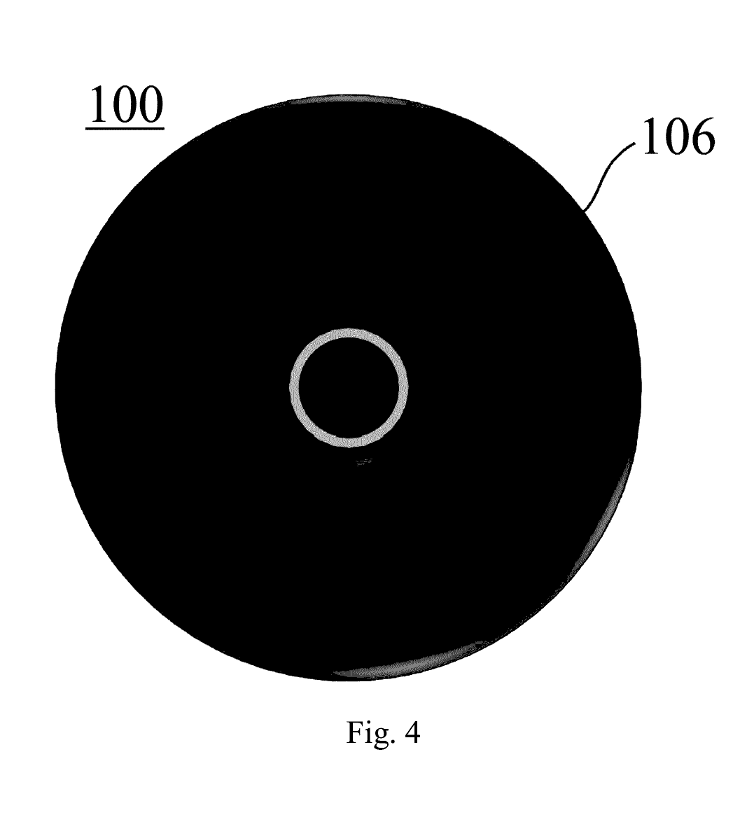

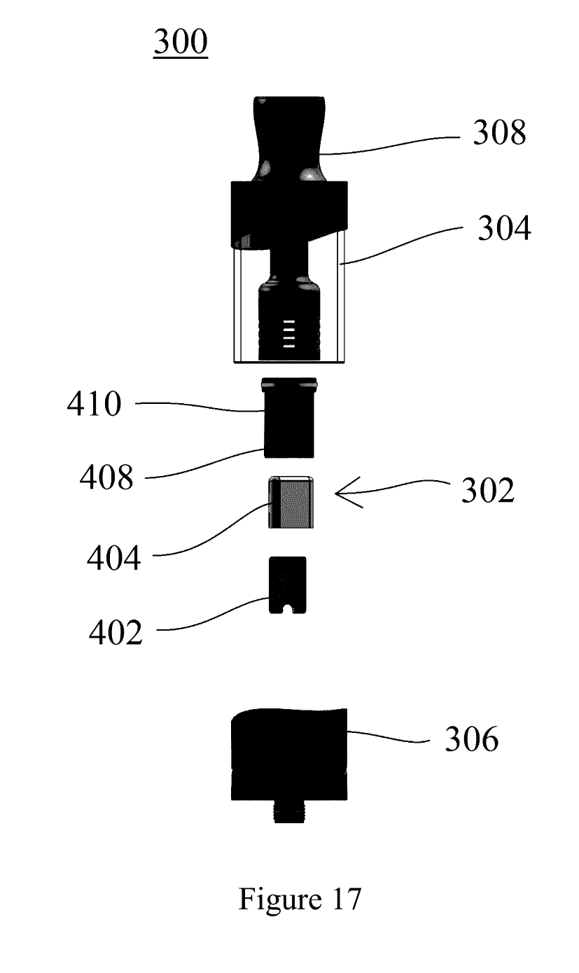

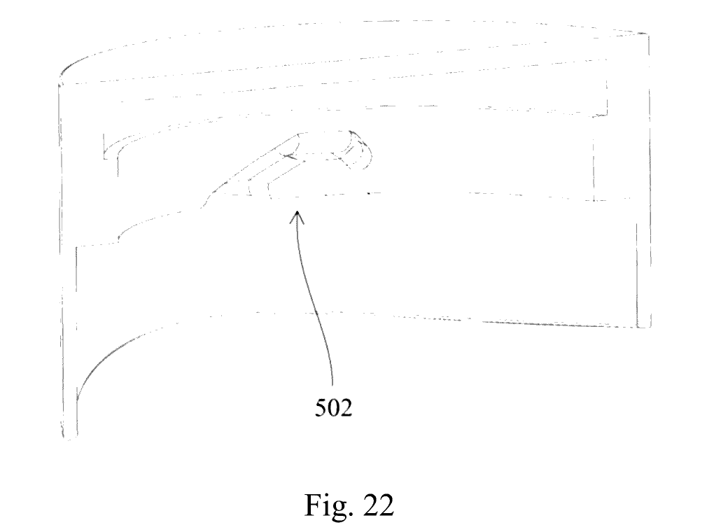

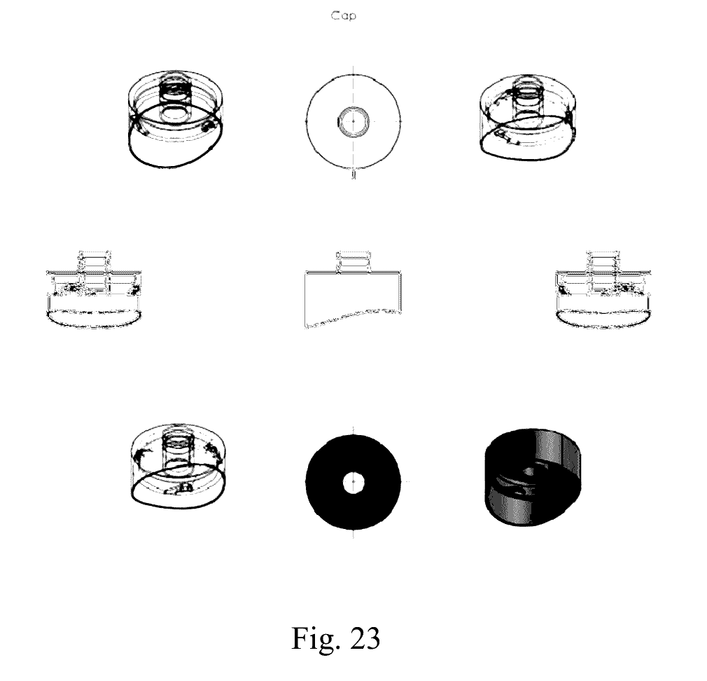

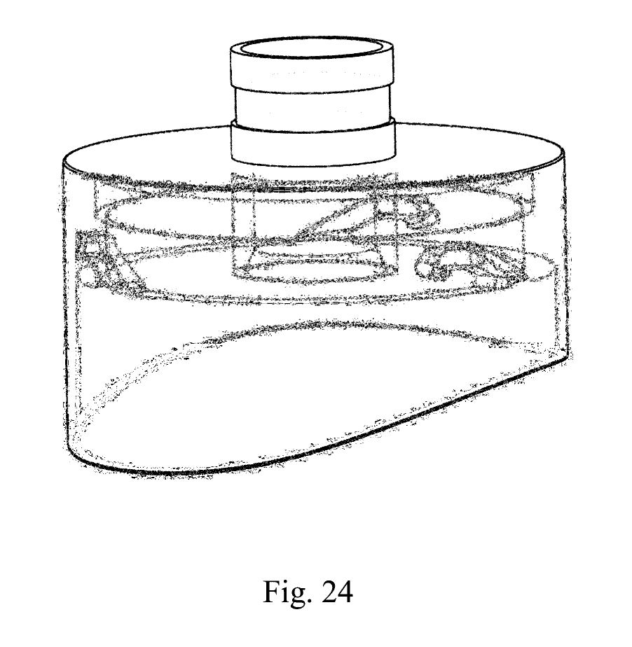

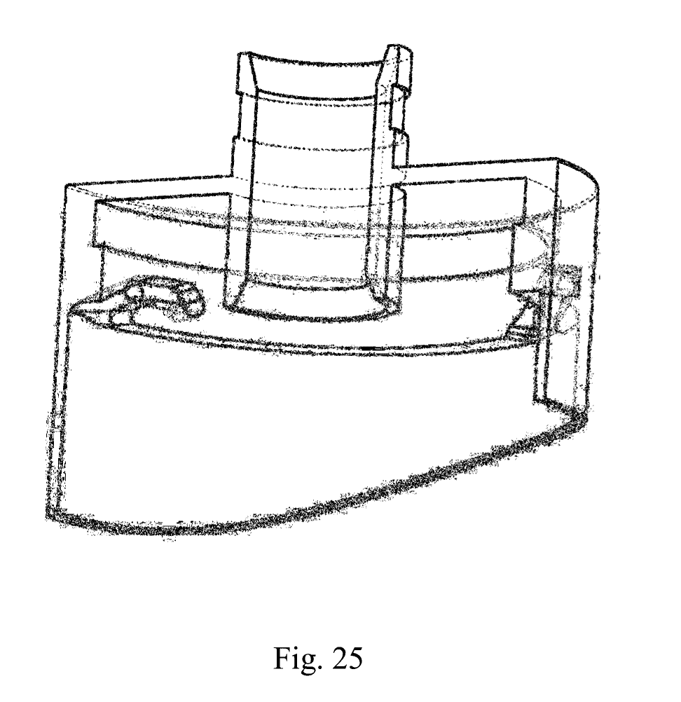

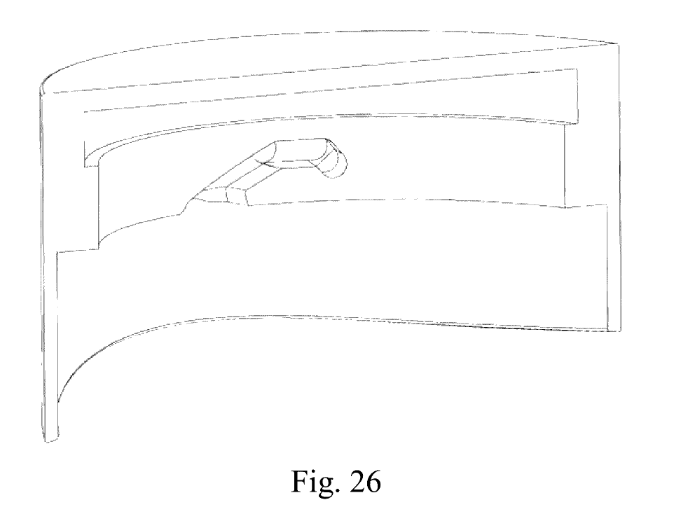

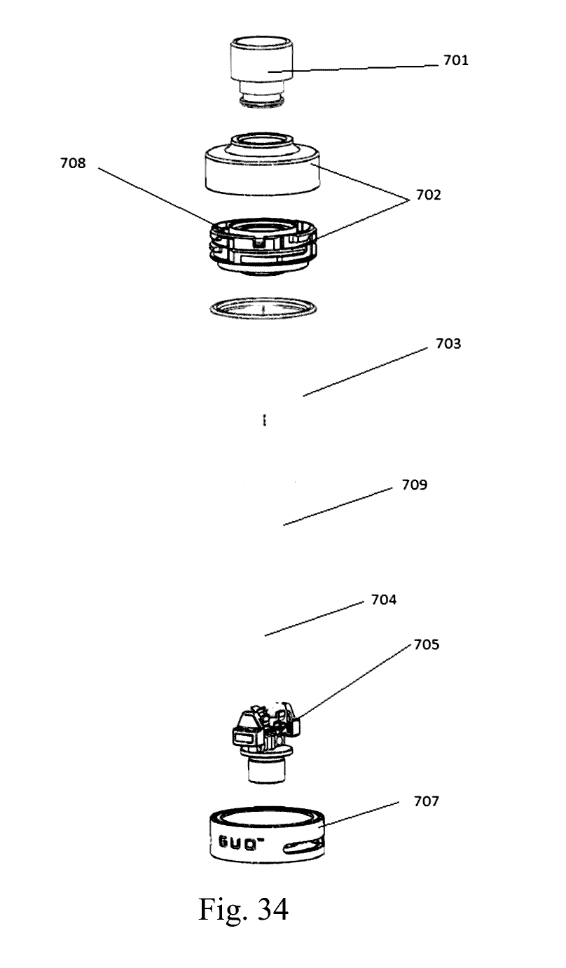

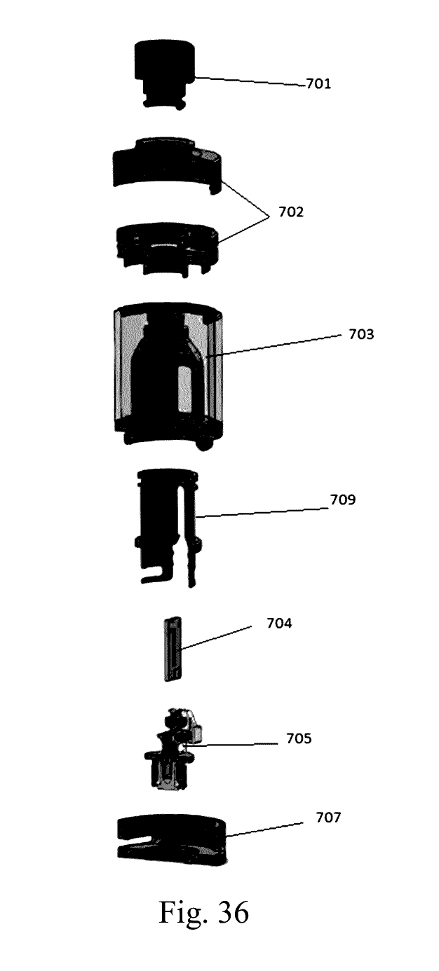

The description will be made as to the embodiments of the present disclosure in conjunction with the accompanying FIGS. 1 through 39. In accordance with the purposes of this invention, as embodied broadly described herein, this invention in one aspect, relates to an atomizer to be used with electronic cigarettes and personal vaporizers.

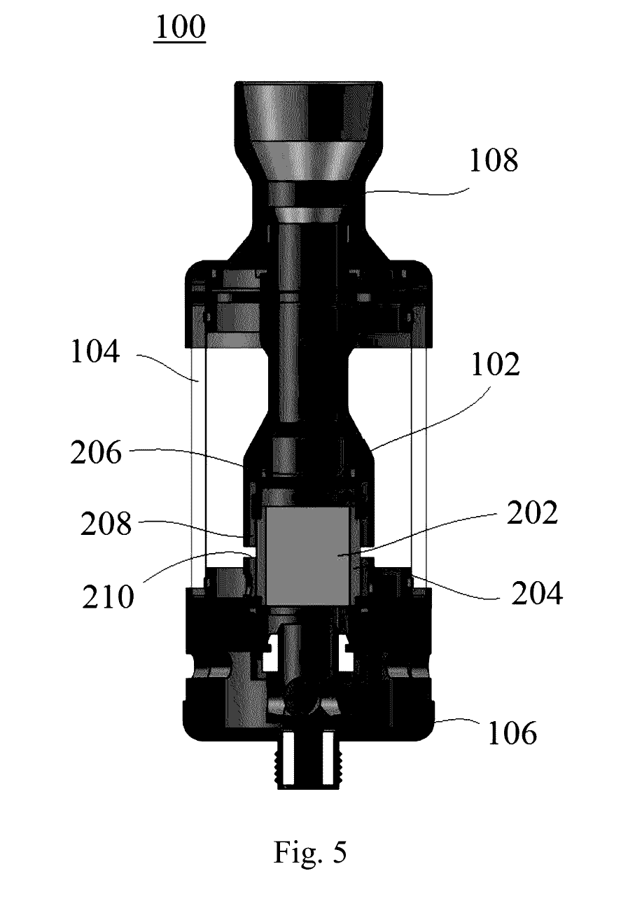

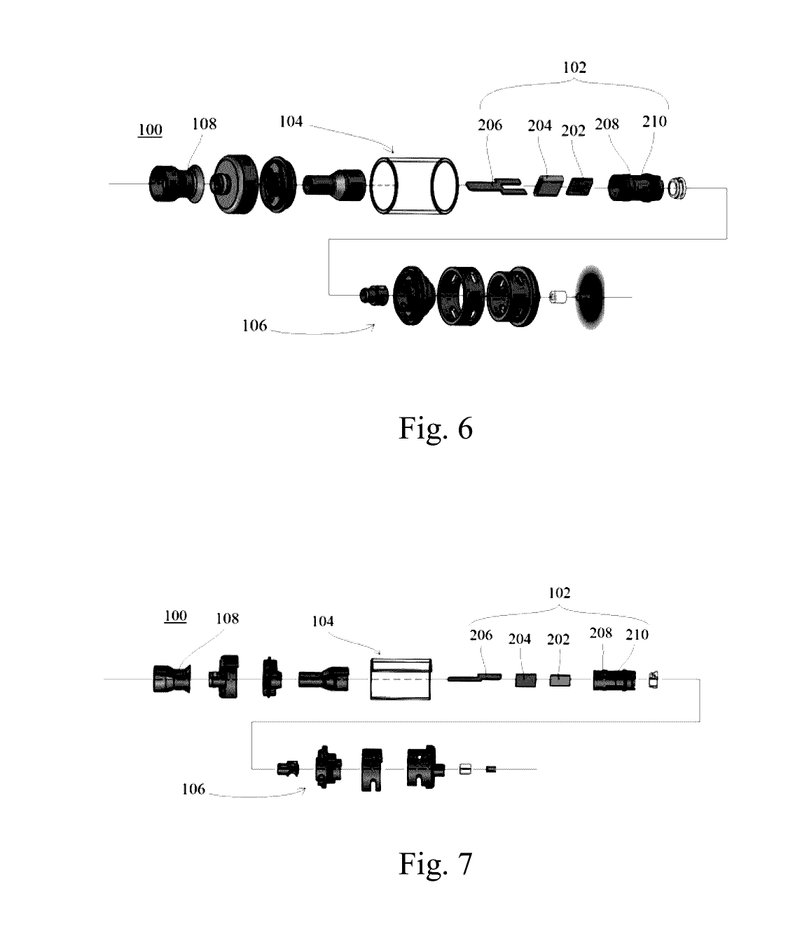

In certain embodiments as shown in FIGS. 1 through 5, an atomizer 100 may include: a housing 102, a chamber 104, a base 106, and an inhaler 108. In one embodiment, the chamber 104 is adapted to receive and store an amount of e-liquid. The base 106 supports the housing 102 and the chamber 104 and contains conductive elements to transmit electrical power sourced from a power source (not shown) of the electronic cigarette/vaporizer to the atomizer 100. The atomizer 100 can connect to a power source (not shown) as is typically known in the prior art. The power source can be a boxy battery pack or a cylindrical housing with rechargeable batteries enclosed within. The inhaler 108 covers the housing 102 and the chamber 104 and guides the generated vapor of the e-liquid outwards. Furthermore, a perspective-exploded view and a cutaway-exploded view of said atomizer 100 are shown by FIGS. 6 and 7. Referring to FIGS. 5-7, it is shown that the housing 102 may be received in the chamber 104 to be in contact with the e-liquid received in the chamber 104, which is also a liquid container in certain embodiments. The housing can have a heating element (to be described below) enclosed in the housing 102 and thus not in direct contact with the e-liquid stored in the chamber 104. The e-liquid can comprise one or more flavors, medicaments, nicotine or non-nicotine liquids or any other inhalable material.

The chamber 104 can be generally sealed such that the e-liquid contained therein does not leak out while a user is using the atomizer 100. As will be described later, a dripper model is also contemplated where the atomizer 100 does not require a chamber 104.

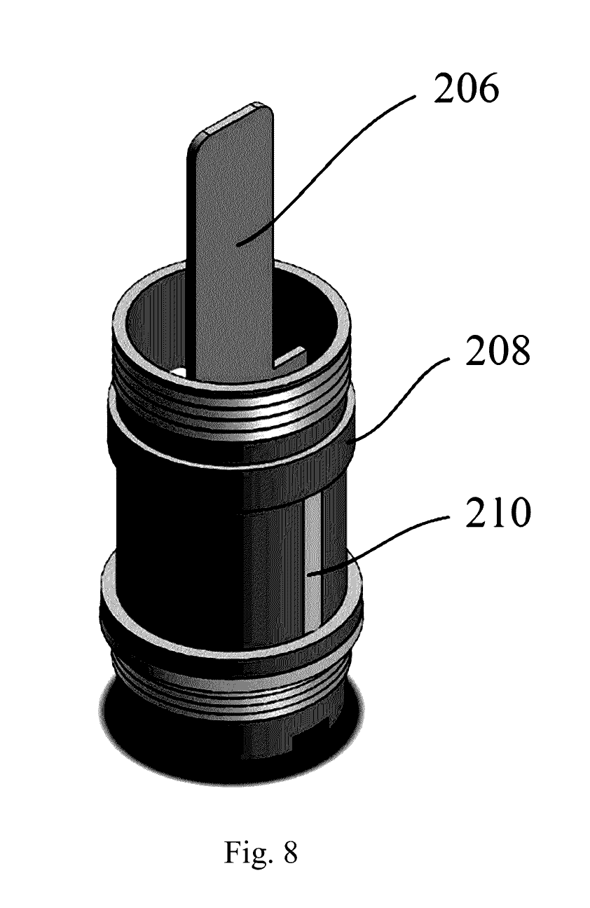

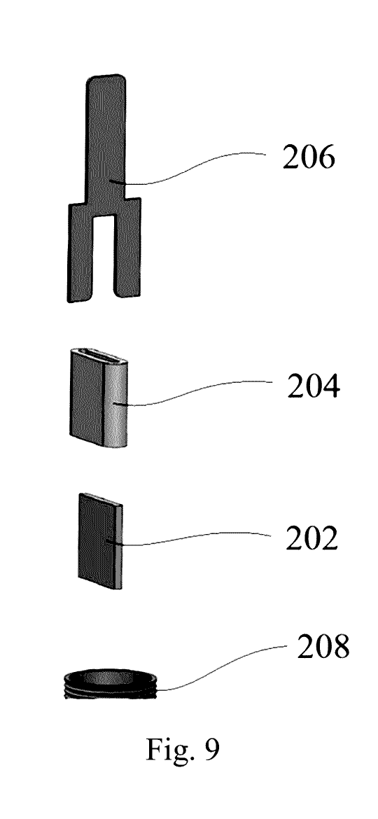

Referring to FIGS. 8 and 9, a perspective view and an exploded-perspective view of the inside of the housing 208 are shown, and the housing 208 can include a heating element 202, a liquid absorber 204, and a holder 206. The heating element 202 electrically connects to a power source, such as a battery, via the base 106 so as to generate heat. The liquid absorber 204 can wrap around the ceramic heating element 202. The inside of the housing 208 is typically not flooded with e-liquid. Instead, the e-liquid stored in the chamber 104 slowly seeps into the inside of housing 208 via the liquid absorber 204 by capillary action. In FIG. 8, a portion of the liquid absorber 204 is exposed to the chamber 104 via opening 210. In this way, e-liquid passes from the chamber 104 through the opening 210 and saturates the liquid absorber 204 (e.g., a sleeve as shown in FIG. 9), which can be heated by the heating element 202 to vaporize the e-liquid.

The liquid absorber 204 can be made from a variety of materials. A liquid absorber 204 can be used in certain embodiments to transport one or more liquid compositions from a chamber 104 to the heating element 202 in the atomizer.

A liquid absorber 204 for use according to one contemplated embodiment of the invention thus can be any material that provides sufficient wicking action to transport one or more liquid compositions to the heating element 204. Non-limiting examples include natural and synthetic fibers, such as cotton, cellulose, polyesters, polyamides, polylactic acids, glass fibers, combinations thereof, and the like. Other exemplary materials that can be used in wicks include fibrous materials, such as polymers, plastics, metals, ceramics, fibers, carbonized filaments, porous materials, porous ceramics, porous aluminum nitride, porous PEEK, porous metals or any other porous material. The liquid absorber further can be coated with materials that alter the capillary action of the fibers. Fibers used in forming the liquid absorber can be provided singly, bundled, as a woven fibrous wick (including meshes and braids), a woven fabric, or as a non-woven fabric. Porosity of the liquid absorber material also can be controlled to alter the capillary action of the liquid absorber including controlling average pore size and total porosity, controlling liquid absorber geometry, and controlling surface characteristics. Separate liquid absorbers 204 also can have different lengths and sizes. The term liquid absorber 204 is also intended to encompass capillary tubes, and any combination of elements providing the desired capillary action can be used.

While the liquid absorber 204 is mainly shown in the configuration of a pre-formed sleeve as shown in FIG. 9, the atomized 100 as disclosed can also accommodate appropriately sized and shaped cotton pieces that a user manually cuts up and wraps around the heating element 202. For example, in Fig., a user may cut up a flat cotton pad and wrap it around the heating element 202. And when the holder 206 closes down towards the heating element 202, the holder 204 can hold the cotton pad against the heating element 202.

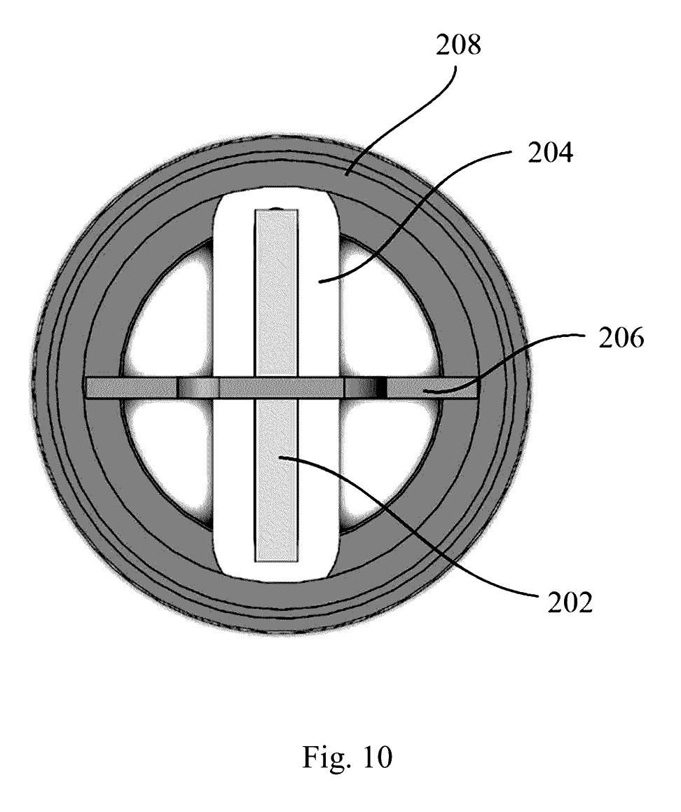

FIG. 10 shows one embodiment from a top view where the liquid absorber 204 can wrap around the heating element 202, while the holder biases the liquid absorber 204 against the heating element 202.

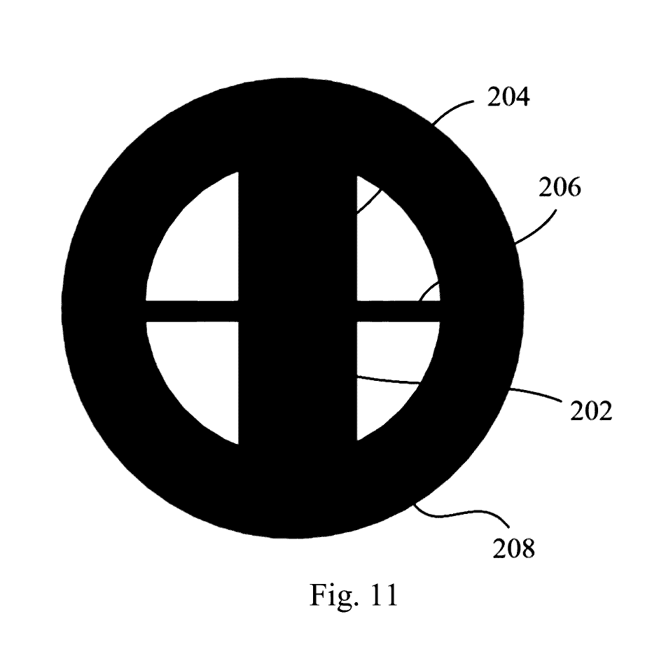

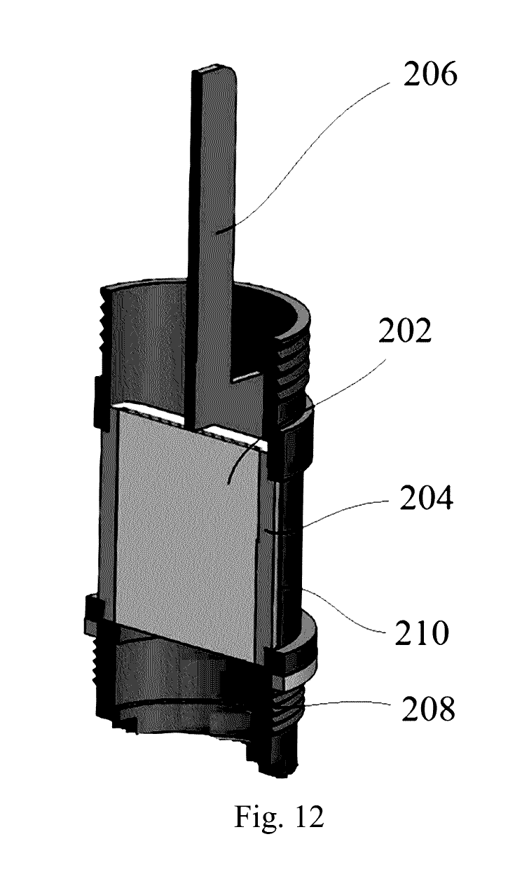

In FIG. 12, the holder 206 can secure the liquid absorber 204 and press it against the heating element 202 to secure them together in the housing 208, and the holder 206 can be electrically separated from the heating element 202. As discussed above, the housing 208 has openings 210 communicating the inner and outer of the housing 208 for the e-liquid to enter and to be absorbed by the liquid absorber 204, and the inner of the housing 208 also communicates with the inhaler 108. Moreover, the housing 208 can be disengaged from the chamber 104 and the base 106 and can be disassembled. In other words, the heating element 202, the liquid absorber 204, and the holder 206 can be detachable from the housing 208, so that the user may easily replace the liquid absorber 204 by a new one if it has been damaged by the heat of the heating element 202. Top view and bottom view of the atomizer are further shown in FIGS. 10 and 11 for clearly illustrating the structure of the atomizer FIG. 12 showing a cutaway view of the housing 208 and its contemplated components. Particularly, the liquid absorber 204 can be wrapped around the heating element 202 and it can also block the openings 210 of the housing 208. Therefore, the liquid absorber 204 can easily absorb e-liquid about the openings 210. In another embodiment, the liquid absorber 204 also can prevent the e-liquid from accessing the electrical circuit connecting with the heating element 202. As will be discussed later, the contemplated chamber 104 may or may not be air sealed.

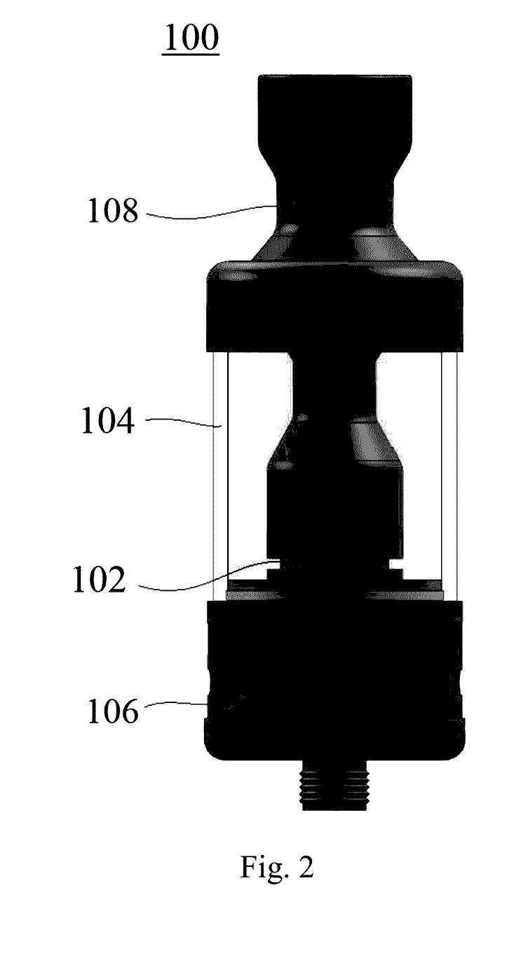

The embodiment as shown in FIGS. 1 and 2 shows a housing 102 with openings that are shaped and located differently from opening 210 of FIG. 8. In FIG. 1, the openings are circular. In FIG. 2, the openings are not shown, but the idea is to have some openings to allow the passage of e-liquid via the liquid absorber 204 which can be disposed across or through the contemplated openings. Other prior art designs on such openings can also be implemented in the disclosed atomizer 100.

It should be noted that the holder 206 should not limit the invention to any particular type or shape of clamping mechanism. The inverse Y-shaped clamp 206 (see FIG. 9) discussed herein can be implemented by various simpler or more complicated holding, fastening, or securing ways. For example, the holder 206 can or cannot be coupled to the base 208 and extend towards the heating element 202 such that the holder 206 has a contact surface to abut the liquid absorber 204 against at least one side of the heating element 202.

In some particular embodiments, a holder 206 may not be necessary at all. For example, a preformed liquid absorber sleeve 204 (as shown in FIG. 9) can slide over the heating element 202, allowing the heating element 202 to heat the e-liquid contained in the sleeve 204, without any abutment from any structural element.

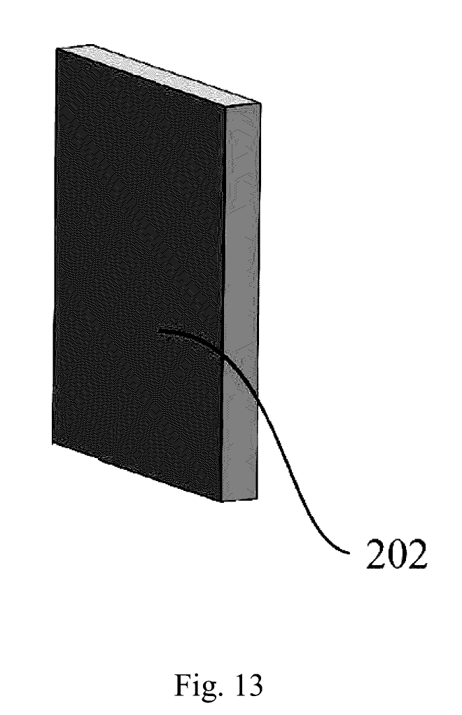

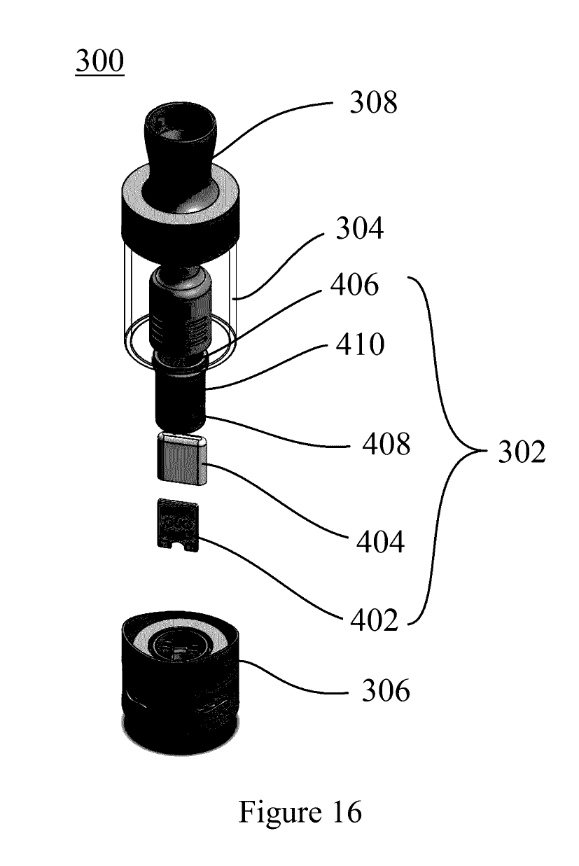

FIG. 13 shows an embodiment of the heating element 202. In this particular embodiment, the heating element has a flat configuration. With said flat configuration, the heating element 202 may have a large vapor production. Furthermore, the heating element 202 may have at least one electrical contact (as shown in FIGS. 16-18, 37, 38, and 39) capable of connecting to a power source.

In certain embodiments, the heating element is made of ceramic materials. In operation, when a user actuates the electronic cigarette/vaporizer, the heating element 202 of the atomizer is heated and thus vaporizes the e-liquid absorbed by the liquid absorber 204 so as to generate vapor of the e-liquid for the inhaler 108 to guide it outwards, thus the user can enjoy the vaporized e-liquid thereby. Specifically, since the heating element 202 can be made of non-metal, the generated vapor will not contain metal particles during operation of the electronic cigarette/vaporizer. Furthermore, even though the holder 206 mayor may not be made of metal, a metal holder 206 will not be heated to a high temperature since the clamp 206 is electrically separated from the heating element 202 and the liquid absorber 204 is physically inserted between the holder 206 and the heating element 202. Therefore, the holder 206 does not undesirably release metal particles into the vapor during operation of the electronic cigarette/vaporizer. As a result, the heavy metal poisoning caused by the conventional electronic cigarette/vaporizer can be minimized.

It is also contemplated that the holder can be a protruding structure such as a rod, a plate, a notch, and a block. Such protruding structure can or cannot be movable, and can have an abutting surface to bias the liquid absorber against the heating element. It is ideally to have an abutting surface spaced apart from the heating element sufficiently to allow the liquid absorber to fit snugly therebetween. In some embodiments, the holder can be adjustable, allowing a user to adjust the gap between the heating element and the abutting surface.

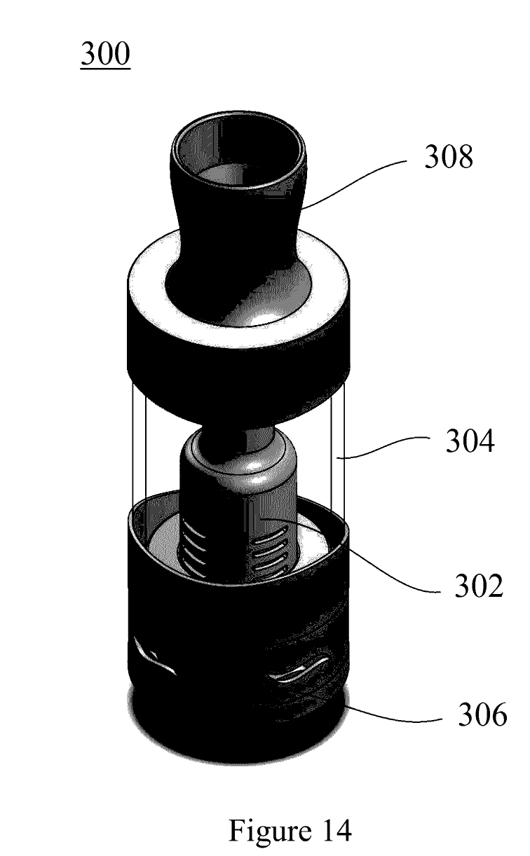

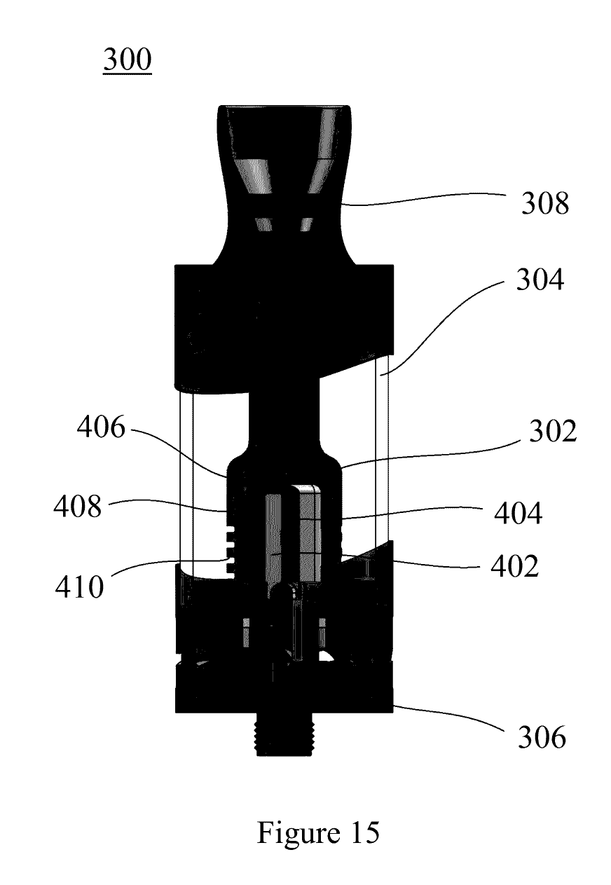

In certain embodiments, as shown in FIGS. 14-17, the atomizer includes an inhaler 308 detachably connected to a chamber 304, a housing 408 (see FIGS. 15 and 16) with a holder 406 disposed within. There can be openings 410 on said housing for the same purpose as discussed above in other contemplated openings 210 (of FIG. 8). Also included are a liquid absorber 404, a heating element 402, electrical contacts and securing means 406 and a base 306 supporting the chamber.

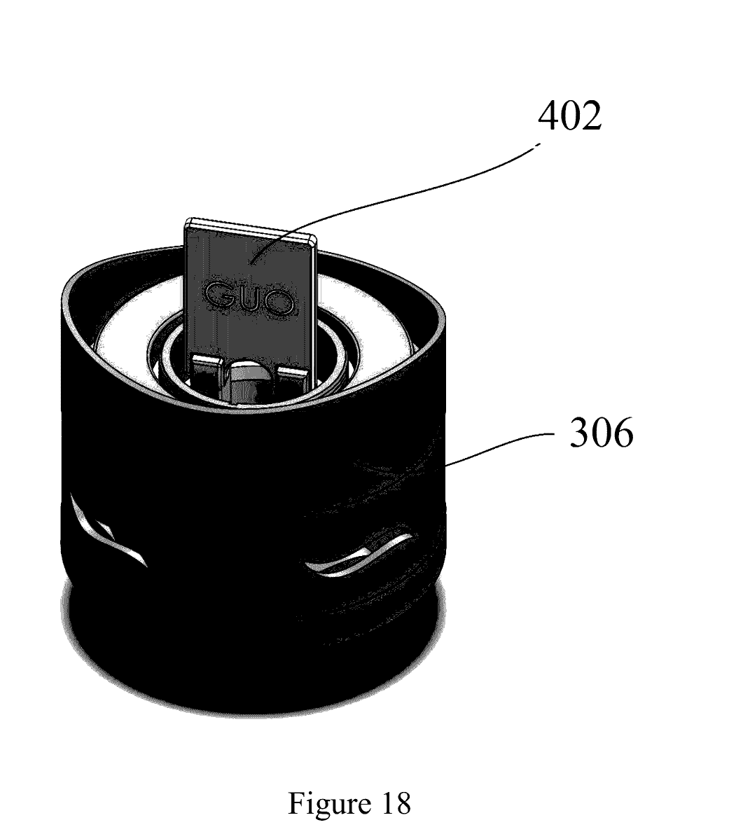

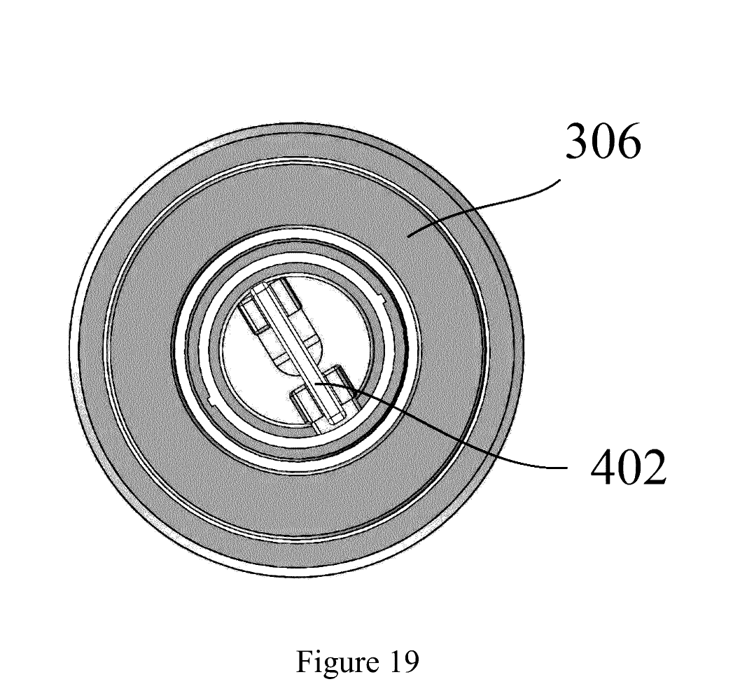

In certain embodiments, as shown in FIGS. 18 through 20, the heating element 402 is disposed on and in direct contact with a receiver inside the base 306. The heating element 402 can have one or more electrical contacts which connect to electrical contacts within the receiver. The electrical contacts on the receiver can also serve as a way of securing the heating element to the receiver. In certain embodiments, the contacts on the receiver (see FIG. 18) can be clamps, resilient leaf springs, fasteners, or any securing structure capable of being electrically conductive and having some biasing force to hold the heating element 402 in place. The receiver may have one or more contacts, and similarly, the heating element 402 might also comprise one of more electrical contacts. In one embodiment, the electrical contacts of the heating element 402 might be on one or two faces of the heating element or hidden within the heating elements. The heating element might also have one or more electrical contacts. These contacts can be in any shape such as polygonal contacts, circular ones or the like.

In FIG. 19, a top view into the base 306 shows a total of four electrical contacts on the receiver. They can be two opposing pairs clamping the heating element 402 on its two bottom terminal ends (more clearly illustrated in FIG. 18).

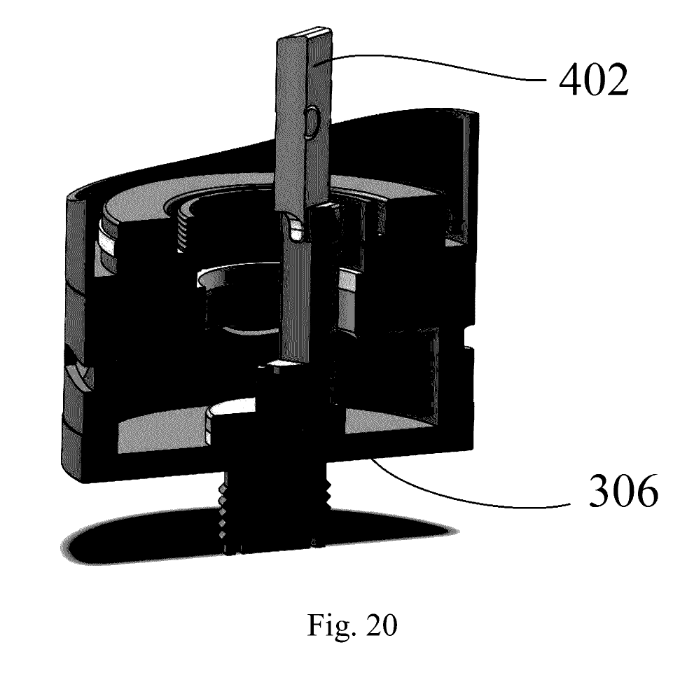

FIG. 20 shows a dissected view of some of the inner components discussed above.

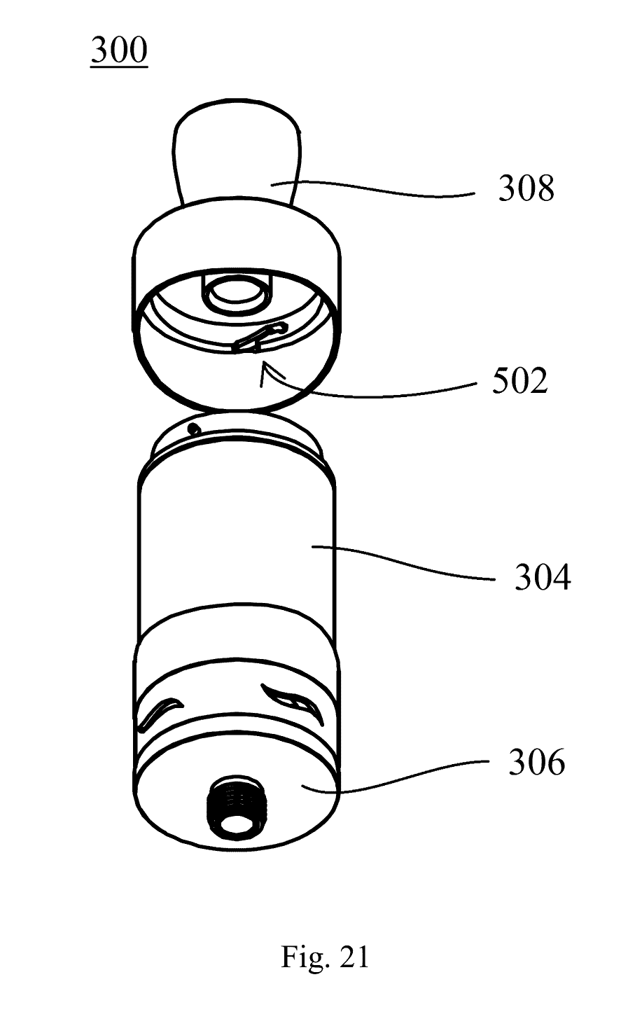

FIGS. 21-26 illustrate an embodiment of a spin and seal locking cap and its various components. These components are optional and can be implemented in various embodiments of the disclosure. These components allow the user to refill e-liquid into the chamber 304 by ways of detaching the inhaler 308 while avoiding overflow or leaks of the liquid. In one embodiment, the atomizer comprises a top fill assembly defined by the connection of the chamber to the inhaler in a spin and seal form. In one embodiment, the inhaler is pivotably coupled to the chamber, and can rotate to pivot sideways, thereby exposing the top of the chamber. In yet another embodiment, the inhaler can flip up, thereby pivoting upwards to expose the top of the chamber. In another embodiment, the inhaler 308 can comprise a central spring and a plurality of grooves engageable with indents located on the neck of the chamber. In yet another embodiment, a seal to keep the system locked can be located on the chamber, on the inhaler side or on both the chamber and inhaler side. In certain embodiments, said seal can be made of silicone or similar material, and can comprise a hole for easy refills.

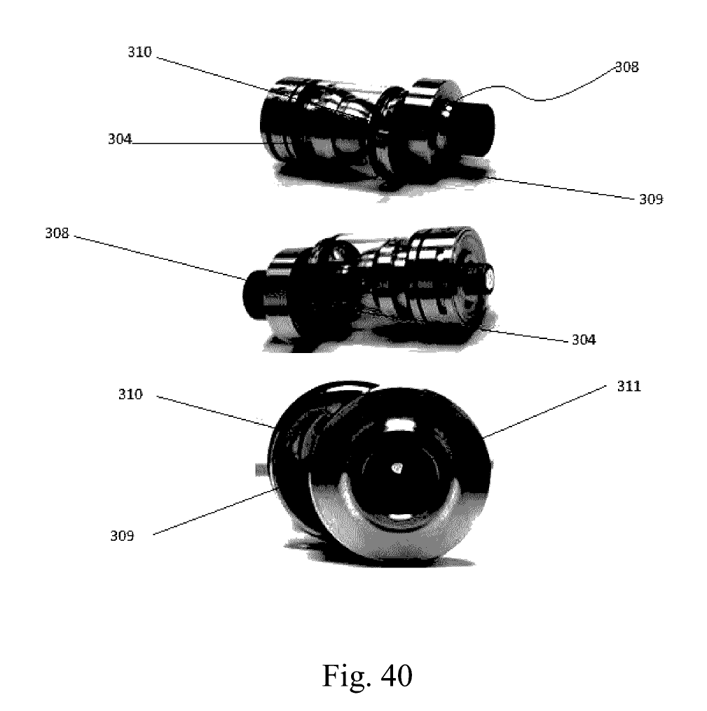

FIG. 40 illustrates another embodiment of the spin and seal feature. This feature allows for an easy fill of the chamber while avoiding leaks. The seal 309 can be made of rubber, silicone, thermoplastics, nitride rubbers, silicone rubber, elastomers, polytetrafluoroethylene, fluoro rubber, leather, felt or a combination thereof. The seal solves leak issues encountered by users when the chamber 304 is open where air flows through the chamber causing the liquid to leak through the base 306 of the atomizer.

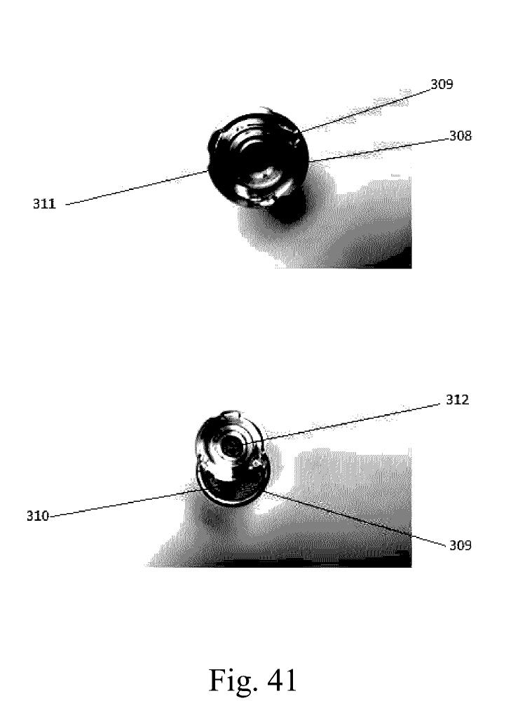

FIG. 41 shows different views of part of the inhaler 308, the silicone seal 309 and the chamber 304. The first view illustrates part of the inhaler 308 in a closed position over the chamber 304 with the silicone seal 109 therebetween. The second view of FIG. 41 illustrates the inhaler 308 pivoted away from the atomizer's central axis. The presence of at least one seal in the these embodiments make it such that when the inhaler and top part of the atomizer is pivoted away from the chamber, the seal 309 prevents air from flooding the chamber and helps avoid leaks at the base. In one embodiment, the seal may look like a sheet of silicon disposed on the chamber side, and can have three openings. The first opening 310 is the fill port, connecting outside space to the inside of the chamber. When the inhaler rotatably pivots away from the atomizer's center axis, the first opening 310 is exposed (see FIG. 41), allowing a user to pour e-liquid into the chamber. The second opening 311 can be centrally located on the sheet of silicon, providing fluid communication and passage of vapor from inside of the base 306 upwards through the center channel of the inhaler 308 and then to the atmosphere. The inhaler can have a flat bottom surface that blocks this second opening 312 when the inhaler is rotatably pivoted open as shown in FIG. 41. In this way, air flow into the interior of the base is severely limited because the top ingress/egress is effectively sealed by the inhaler 308 disposed over on top of the second opening 311. The optional third opening 312 can be disposed on the sheet of silicon seal and allows passage of air from the chamber to the atmosphere. When the inhaler pivots open as shown in FIG. 41, the third opening 312 is exposed, so that when e-liquid is pour though the first opening 310, air exits from the chamber 304 through the third opening 312. In one general concept, the first opening can be disposed adjacent to the edge of the seal, such that when the user pivots the inhaler away from the chamber, the bottom of the inhaler covers over the vapor egress of the base and the first opening is on the visible side allowing the user to refill the chamber with liquid. The inhaler can be pivotably attached to the chamber 304 in various ways.

In a certain embodiment, the chamber has a top tubular portion rotatably engageable to a lower tubular portion; a plurality of engageable indents on said top tubular portion capable of engaging with the grooves of said inhaler; the top tubular portion comprises a strip hollowing with a first end and a second end; and the lower tubular portion comprises a protrusion engageable with said hollowing of said top tubular portion. When the inhaler is connected to the top tubular portion, and the user rotates the top tubular portion around the lower tubular portion and the protrusion from the lower tubular portion is aligned with the first end of the top tubular hollowing and pushes the inhaler on the lower tubular portion the top fill assembly is locked, and when and the protrusion from the lower tubular portion is aligned with said second end of said top tubular hollowing and rotates the inhaler the top fill assembly is opened allowing the user to fill the liquid container with liquid while preventing flooding.

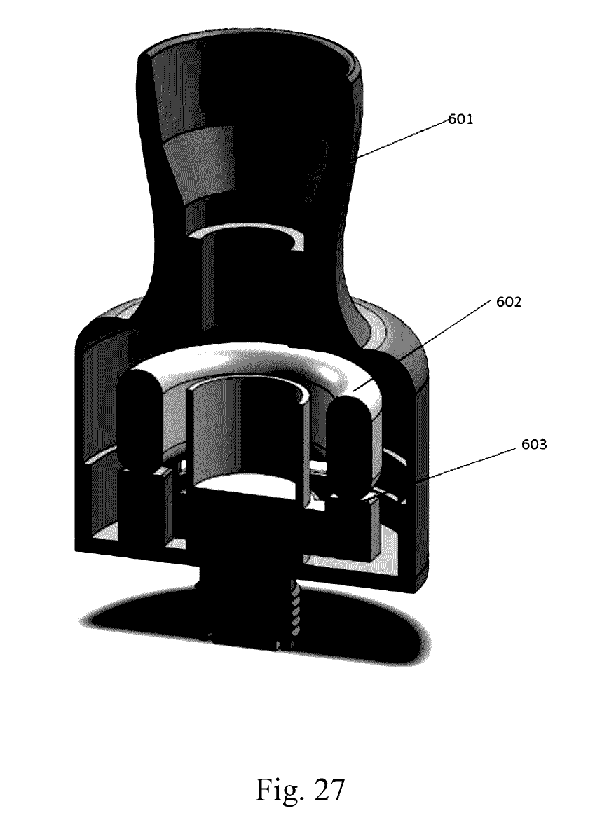

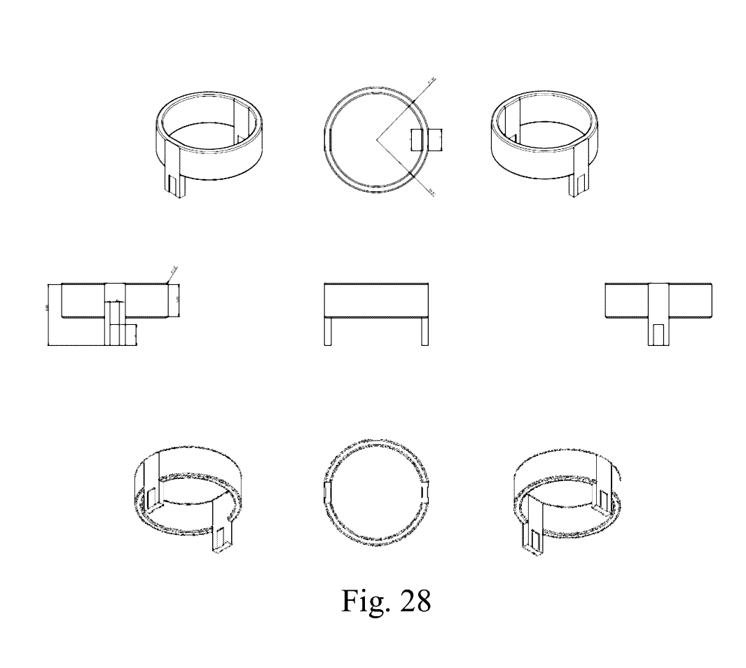

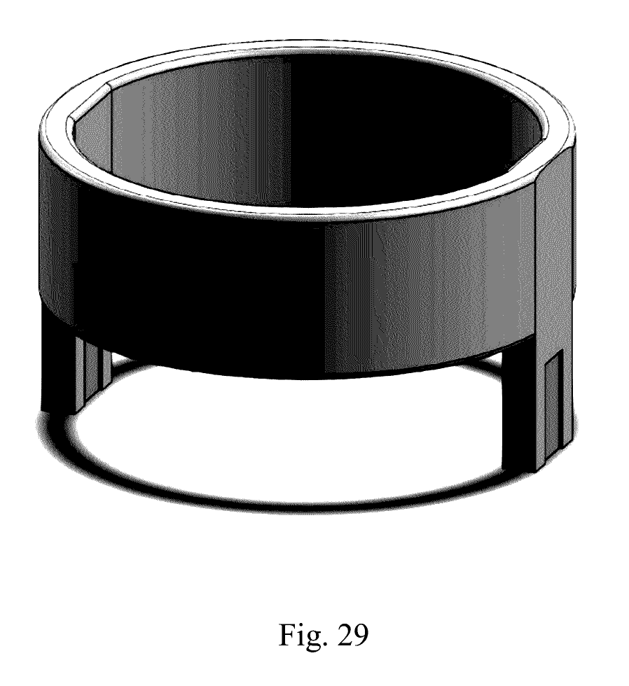

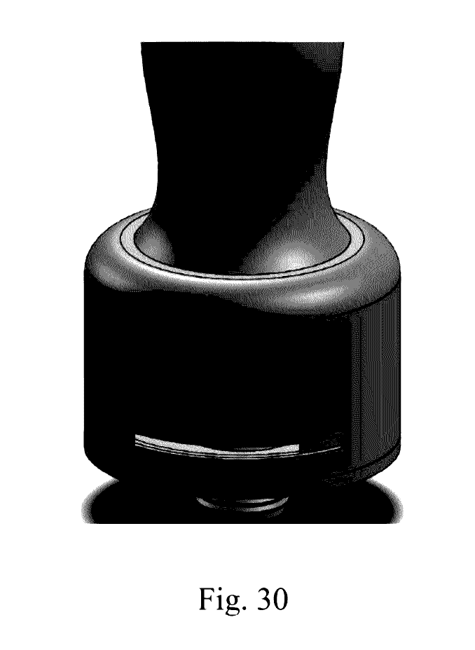

As for FIGS. 27-32, various designs of a dripper-type atomizer are disclosed. The dripper-type atomizer is one that does not require a tank to hold and store liquid. In certain embodiments, the dripper chamber is sealed. However, other dripper atomizer embodiments may not be sealed. FIG. 27 shows a cross sectional view of an atomizer with a dripper embodiment. The dripper atomizer includes: an inhaler 601, a circular liquid absorber 602, a circular heating element 603, a receiver 604 and a power connection 605. FIGS. 28 and 29 show a certain embodiment of a circular heating element. The heating element depicted in FIGS. 28 and 29 show an annular body, however, the body can be annular, rectangular, square, a polygon, with or without hollowing in the middle. The heating elements are shown in FIGS. 28 and 29 having electrically conductive contacts located on two downwardly extending legs. These legs can insert into corresponding receiving structure on a base of the atomizer FIG. 30 show a perspective view of a dripper component with threading engageable with the battery portion of an electronic cigarette.

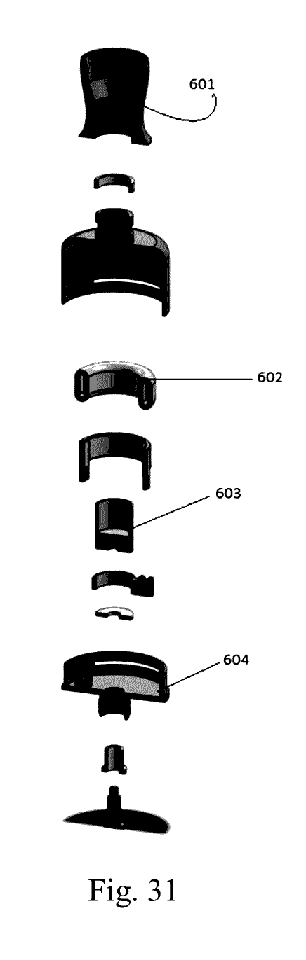

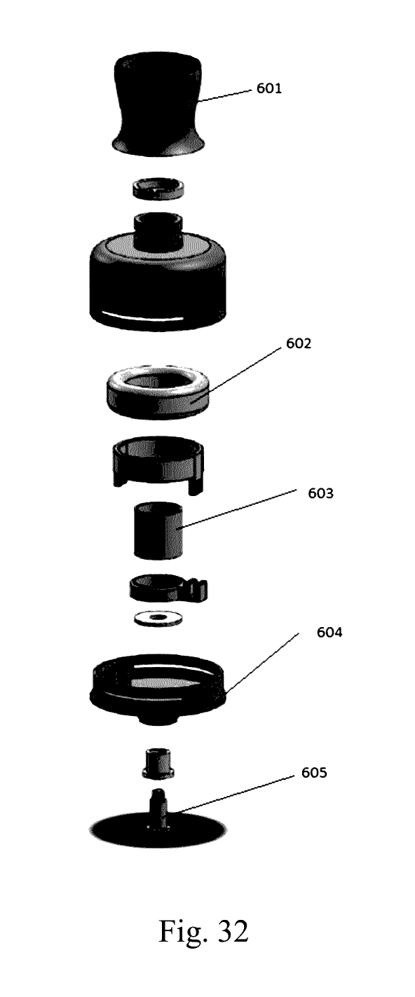

FIGS. 31 and 32 show an exploded cross sectional view of a dripper atomizer with components. The dripper atomizer includes: an inhaler 601, a liquid absorber 602, a circular heating element, a receiver 604 and a power connection 605. The receiver 604 supports the heating element and the liquid absorber 602 and transmits electrical power sourced from a power source of the electronic cigarette/vaporizer to the heating element via the power connection 605.

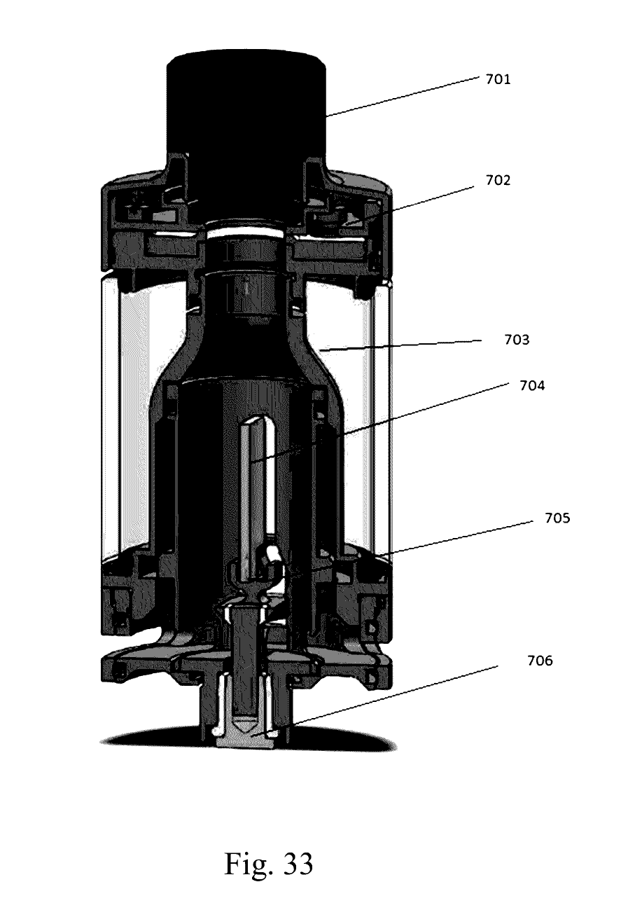

In a certain embodiment as shown in FIGS. 33 through 36, the atomizer includes: an inhaler 701, a spin and seal top fill 702, a chamber 703, a heating element 704, a receiver 705, a power connection 706, a base 707, a seal 708, and a housing 709. The inhaler 701 can be removably attached to the spin and seal top fill 702. Part of the spin and seal 702 is attached to the chamber 703, and a housing 709 is removably attached to the receiver 705. The base 707 can comprise air vents.

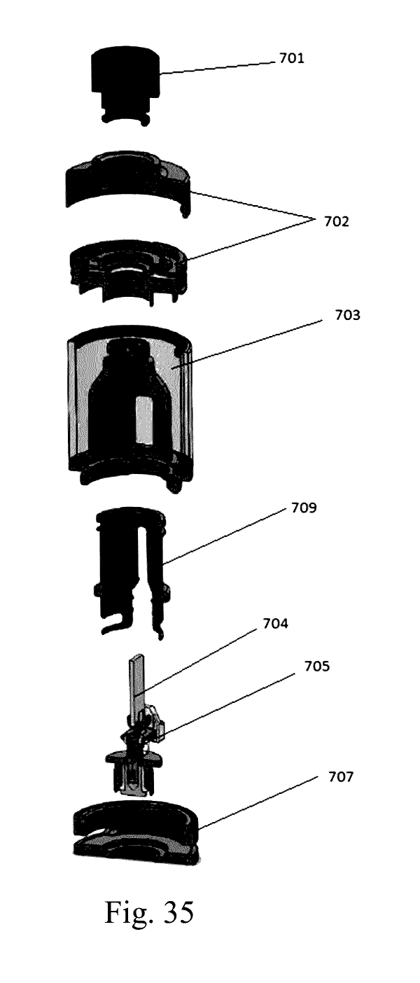

In FIG. 35, a holder within the housing 709 secures a liquid absorber and the heating element together. The holder within the housing is electrically separated from the heating element. The housing 709 has openings communicating the inner and outer of the housing 709 for the e-liquid to enter and to be absorbed by a liquid absorber, and the housing 709 also communicates with the inhaler 701. Moreover, the housing 709 can be disengaged from the chamber 703 and the base 707 and can be disassembled. In other words, the heating element 704, and the chamber 703 are detachable so that the user may easily replace, clean or customize components of the atomizer. The chamber can comprise at least one sealing ring to prevent leaks, and can be coupled to the base.

The base 707 may comprise air vents. In one embodiment, the base can include an air adjustment assembly having an outer tubular ring rotatably engaged to an inner tubular portion, where the outer tubular ring has first air vents, and the inner tubular portion has second air vent. When a user rotates the outer tubular ring around the inner tubular portion of the air adjustment assembly, and when the location of the second air vents match the location of the first air vents, air flows from outside to the air adjustment assembly. And when the user further rotates the outer tubular ring around the inner tubular portion, the air flow decreases, and when the locations of the first and second air vents completely misalign, the air flow stops.

The chamber 703 of the atomizer can be formed of any material suitable for forming and maintaining an appropriate conformation, such as a tubular shape, and for retaining therein the suitable components of the article. The chamber can be formed of a single wall, as shown in FIG. 35. In some embodiments, the chamber can be formed of a material (natural or synthetic) that is heat resistant so as to retain its structural integrity—e.g., does not degrade—at least at a temperature that is the heating temperature provided by the resistive heating element, as further discussed herein. In some embodiments, a heat resistant polymer or a metal (e.g., stainless steel) may be used. In other embodiments, the chamber can be transparent. Ceramic materials may also be used.

An atomizer according to the invention comprises a heating element that heats a liquid to produce a vapor for inhalation by a user. An atomizer as described herein can be particularly characterized by comprising a microheater as a heating element. Specifically, the heating element can be in electrical connection with an electrical power source, as further described herein. The atomizer can include only a single heating element. In other embodiments, however, the smoking article can comprise a plurality of heating elements. Thus, it is understood that although the present disclosure may describe the atomizer in terms of “a” heating element or “the” heating element, the disclosure is meant to encompass embodiments wherein the atomizer includes a plurality of heating elements.

In some embodiments, the heating element used in the presently described smoking article can be characterized as micro-electro-mechanical systems based heater. Such heating element can emit heat by applying an electrical current to a resistor and can provide advantages such as low power input requirement and very short response time. Such heating element is highly advantageous in a smoking article, as presently described, since it can provide for low voltage and/or low power device function.

The heating element used in the presently described atomizer also can be characterized as a thick film heater. Resistors for thick film heaters can be screen and/or stencil printed; the resistive material is a special paste with a mixture of a binder, a carrier, and the oxides to be deposited. The binder is a glassy frit and the carrier exists of organic solvent systems and plasticizers. The resistive layer is printed onto a substrate. After the firing of the paste on the carrier, the film becomes glasslike, which makes it well protected against moisture. Unlike thin film, this process is additive. This means that the resistive layers are added sequentially to the substrate to create the conducting patterns and resistance values.

The heating element and electrically conductive material used in the heating element can comprise essentially any material that is both electrically conductive and suitable for thick film formation. For example, the heating element material can be selected from the group consisting of elemental metals, metal alloys, silicon (including single crystal silicon and poly-silicon), ceramics, nitrides, and combinations thereof. In other embodiments, the electrically conductive material can be formed of platinum, gold, silver, copper, aluminum, tungsten, zinc, nickel, titanium, nichrome, silicon carbide, poly-silicon, single crystal silicon, titanium nitride, and the like. In a particular embodiment materials such as tungsten and aluminum nitrides can be particularly beneficial due to exhibiting similar thermal coefficients, good oxidation resistance and long-term stability. In yet another embodiment, the heating element is made of ceramics, said ceramic material made of tungsten embedded in aluminum nitride.

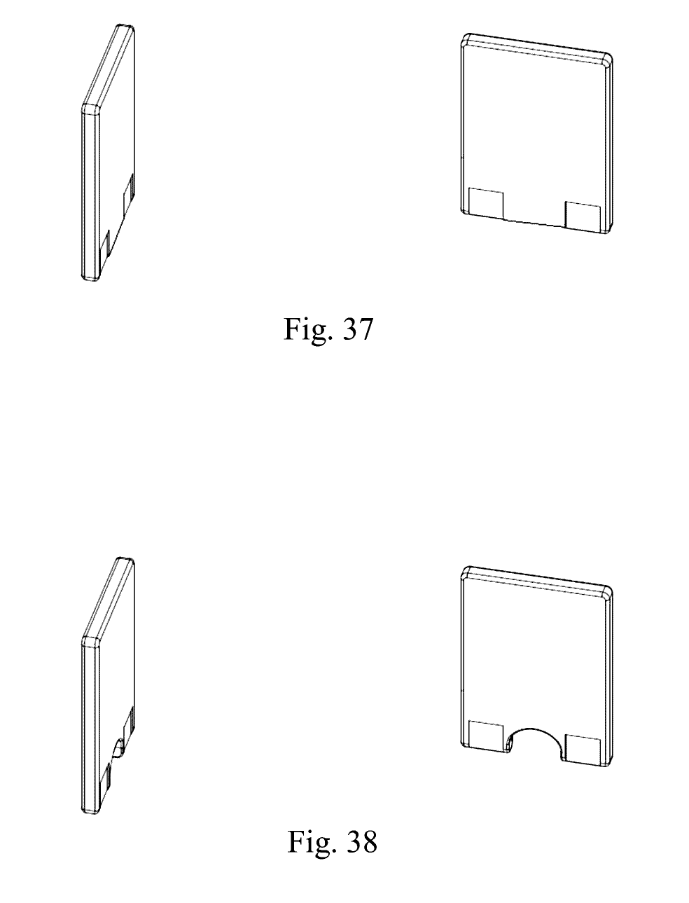

FIGS. 37 through 39 show various embodiments of heating elements. The heating element configuration is not limited to a flat or cylindrical configuration. The heating element may be in the form of a flat configuration, be it a rectangular heating element, a disk, a semi disk or any polygonal shaped heating element. In another embodiment, the atomizer comprises multiple heating elements. FIGS. 37 and 38 also show two electrical contact surfaces on the heating element. In other embodiments, the heating element may comprise at least one contact surface on the face of the heating element, or may comprise contacts on various faces of the heating elements. In yet another embodiment, the heating element may be cylindrical with concentric contact surfaces.

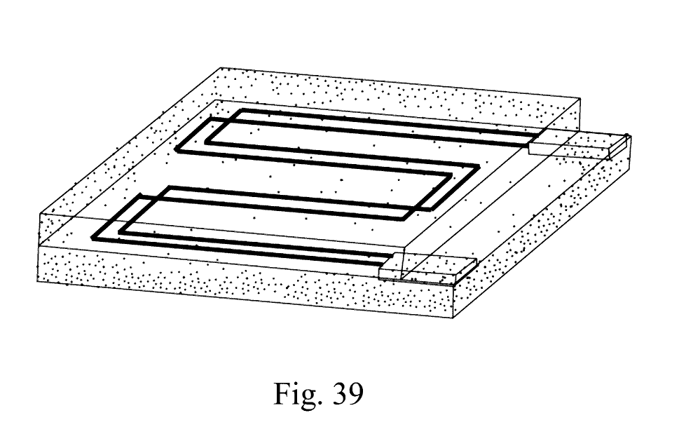

An exemplary embodiment of a heating element that can be used according to the present disclosure is shown in FIG. 39. As seen therein, the heating element can be formed of multiple layers of heating traces within a solid material, wherein the solid material can be in a flat configuration. The heating element can also include electrically conductive contacts as shown in FIG. 37 and FIG. 38 to provide for an electrical connection of the heating element (specifically the electrically conductive material) with the further electrical components of the article described herein, including the various control components and the electrical power source. As shown in FIG. 39, there can be two heating traces embedded in a composite material. When electricity flows through the heating traces via the conductive contacts, the heating element generates heat. The traces can or cannot overlap each other either vertically or horizontally. It some embodiments, they can be designed to have minimum overlap so as to evenly distribute heat to the composite material. Using trace wire to heat a pipe or to heat a flat metallic surface is known. Prior art uses of trace wire do not consider any kind of three dimensional applications of trace wires. In FIG. 39, the two heating traces can be embedded within a three-dimensional solid material, and the two heating traces can be spaced apart. In other embodiments, more than two heating traces can be used.

In sum, with the atomizer, the proposed electronic cigarette/vaporizer can generate e-liquid vapor without metal particles. Therefore, the user of an electronic cigarette/vaporizer with the atomizer can enjoy thee-liquid without suffering from the possibility of heavy metal poisoning. Namely, the provided electronic cigarette/vaporizer can greatly improve safety of e-cigarette/vaporizer usage.

Many alterations and modifications may be made by those having ordinary skill in the art without departing from the spirit and scope of the invention. Therefore, it must be understood that the illustrated embodiments have been set forth only for the purposes of example and that they should not be taken as limiting the invention. For example, notwithstanding the fact that the elements of a claim are set forth below in a certain combination, it must be expressly understood that the embodiment includes other combinations of fewer, more or different elements, which are disclosed herein even when not initially claimed in such combinations.

Thus, specific embodiments and applications of electronic cigarettes and vaporizers atomizers have been disclosed. It should be apparent, however, to those skilled in the art that many more modifications besides those already described are possible without departing from the disclosed concepts herein. The embodiment, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements or components in a non-exclusive manner, indicating that the referenced elements or components, may be present, or utilized, or combined with other elements or components, that are not expressly referenced. Insubstantial changes from the claimed subject matter as viewed by a person with ordinary skill in the art, now known or later devised, are expressly contemplated as being equivalent within the scope of the claims. Therefore, obvious substitutions now or later known to one with ordinary skill in the art are defined to be within the scope of the defined elements. The claims are thus to be understood to include what is specifically illustrated and described above, what is conceptually equivalent, what can be obviously substituted and also what essentially incorporates the essential idea of the embodiment.

What is claimed is:

1. An atomizer comprising:
an inhaler having a first end and a second end through both of which air flow passage partially extends;
a chamber coupled to said second end of said inhaler;
One or more heating elements positioned within the chamber to be in contact with a liquid;
a liquid absorber coupled to the heating element;
a receiver to receive the heating element, wherein the receiver has at least one conductive contact, and the receiver comprises a power connection connectable to a power supply.

2. The atomizer as recited in claim 1, wherein at least one contact is a clamp, or a resilient leaf spring.

3. The atomizer as recited in claim 1, wherein the receiver has at least one fastener to secure the heating element.

4. The atomizer as recited in claim 1, wherein the heating element has at least one electrical contact on an outside surface of the heating element.

5. The atomizer as recited in claim 1, wherein the heating element has at least a pair of electrical contacts on one side of the heating element.

6. The atomizer as recited in claim 5, wherein the heating element has at least a pair of electrical contacts on another side of the heating element opposite to the one side.

7. The atomizer as recited in claim 1, wherein the heating element has a flat configuration.

8. The atomizer as recited in claim 1, wherein the heating element is cylindrical.

9. The atomizer as recited in claim 1, wherein the heating element has multiple layers of traces.

10. The atomizer as recited in claim 9, wherein the multiple layers of traces do not overlap each other in at least some portions.

11. The atomizer as recited in claim 1, wherein the heating element comprises ceramic elements.

12. The atomizer of claim 1, wherein the heating element includes Aluminum Nitride.

13. The atomizer as recited in claim 1, wherein the heating element is a thick film ceramic heater.

14. The atomizer as recited in claim 1, wherein the chamber comprises a holder to bias the liquid absorber against the heating element.

15. The atomizer as recited in claim 14, wherein the holder has contacting surfaces opposite each other on either side of the heating element.

16. The atomizer as recited in claim 14, wherein the holder has a contact surface surrounding the heating element.

17. The atomizer as recited in claim 1, wherein the liquid absorber is at least one of a sleeve, a metallic mesh, a fibrous material and a porous ceramic material.

18. The atomizer as recited in claim 1, wherein the chamber is a sealed container to hold liquid.

19. The atomizer as recited in claim 18, wherein the chamber is defined by a plurality of walls, said chamber including at least one entry configured to allow passage of liquid into said chamber and at least one exit configured to allow passage of formed vapor therefrom.

20. The atomizer as recited in claim 1, wherein the inhaler is pivotably coupled to the chamber.

21. The atomizer as recited in claim 1, wherein the inhaler comprises at least one silicone seal with an opening, wherein the silicone seal is disposed between the inhaler and the chamber.
